(19) United States
(12) Patent Application Publication
(10) Pub. No.: US 2024/0245538 A1
(43) Pub. Date: Jul. 25, 2024

(54) SELF-EXPANDING STENT HAVING STEPPED RADIAL-FORCE PROFILE

(71) Applicant: Angiolutions GmbH, Hannover (DE)

(72) Inventors: Uwe Raaz, Leipzig (DE); Thomas Nissl, Mackenrode (DE); Isabel Nahal Schellinger, Leipzig (DE)

(21) Appl. No.: 18/566,293

(22) PCT Filed: May 6, 2022
(86) PCT No.: PCT/EP2022/062278

§ 371 Date: Dec. 1, 2023

(30) Foreign Application Priority Data
Jun. 4, 2021 (DE) 10 2021 114 444.2
Jun. 4, 2021 (DE) 10 2021 114 450.7

(51) Int. Cl. A61F 2/915

(52) U.S. Cl. CPC A61F 2/915; A61F 2002/91575; A61F 2250/001; A61F 2250/0039; A61F 2250/0048

(57) ABSTRACT

A self-expanding, flexible, intravascular stent for use in a vessel includes at least two annular segments axially connected to each other. Each of the at least two annular segments is formed of a plurality of struts connected to each other in a meandering manner. The stent has a crimped state having a reduced diameter and an expanded state having a nominal diameter. The annular segments have a first expansion characteristic in a first diameter range less than the nominal diameter and a second expansion characteristic, different from the first expansion characteristic, in a second diameter range greater than the nominal diameter.

The invention relates to a self-expanding stent having a plurality of annular segments having a serpentine shape and formed from a plurality of struts, wherein the annular segments are connected to adjacent annular segments. The stent is intended for endovascular applications.

Stents for expanding constricted blood vessels and for anchoring vascular implants in a blood vessel are known and widely described. Differentiation is made between self-expanding stents made of a shape memory alloy and balloon-expanding stents, typically made of a medical steel, both implanted by means of endovascular catheters.

Stents are typically made of a plurality of annular segments connected to adjacent annular segments by means of connecting struts. The annular segments often have a serpentine or zig-zag shape and are formed by struts spread apart from each other during expansion. The individual struts are connected to each other by means of bends. The reduction in length occurring with said technique can be counteracted by a suitable design and/or a suitable arrangement of the connecting struts.

The opening and radial expansion of a blood vessel can require substantial radial forces that must be applied by the corresponding stent. For self-expanding stents, this requires a corresponding size of the struts. For balloon-expanded stents, the radial forces are applied by the balloon used for expanding. The chronic outward force (COF) permanently acting on the wall of the vessel after expansion of a self-expanding stent ensures stable anchoring of the stent at the vessel wall and further acts against the tendency of the vessel to contract (recoil) again.

The COF of a stent is a permanent mechanical load on the vessel wall that can result in compensatory vessel growth processes and a progressive increase in the diameter of the vessel-known as adaptive vessel remodeling. As a result, the solid bond produced after implantation between the vessel and stent becomes loose.

The object is achieved in general by means of a self-expanding stent according to the invention having a stepped (dependent on the degree of expansion of the stent) radial force profile. The stent thereby expands at first with a high, predefined first radial force (F1) until a specific nominal diameter is reached, or to the level of the predefinable first radial force (F1). Past the nominal diameter, the stent allows further expansion until a specific maximum diameter is reached. The difference between the nominal and maximum diameter thereby corresponds to the “expansion reserve” of the stent. When the stent transitions from the nominal diameter thereof into the expansion reserve, the radial force thereof drops abruptly (in a step) to a significantly lower radial force level of a predefinable second radial force (F2) acting for achieving the maximum diameter.

High radial forces/COF (F1) are generated by the implantation at first, in order to achieve stretching of the vessel to a desired target diameter, and closer contact of the stent to the vessel wall is thereby produced in order to prevent migration of the stent, for example, or, for the case of covered stents, to prevent blood flow between the stent and vessel wall (leakage). The nominal diameter of the stent after expanding corresponds to the target diameter of the vessel.

After the stent has grown into the vessel wall, however, loosening of the stent due to further progressive growth of the diameter of the target vessel (adaptive remodeling) is prevented. The stent can therefore indeed follow further vessel growth past the nominal diameter thereof, but said stent exerts only a very low radial force/COF (F2). The stent thus follows a vessel dilation after implantation only with a low intrinsic COF (active) or purely passively and does not stimulate any further vessel growth. The invention proposes a self-expanding, flexible, intravascular stent having at least one, preferably at least two annular segments axially connected to each other and each formed of a plurality of struts connected to each other in a serpentine manner by means of bends. The stent has a crimped state having a reduced diameter and an expanded state having a nominal diameter.

In a first consideration, the annular segments preferably comprise a first expansion characteristic in a first diameter range less than the nominal diameter and a second expansion characteristic, different from the first expansion characteristic, in a second diameter range greater than the nominal diameter.

While conventional self-expanding stents can expand only up to the nominal diameter and can no longer expand along for the case of vessel remodeling above the nominal diameter, a stent able to expand even beyond the nominal diameter is proposed according to the invention. Above the nominal diameter, namely in the second diameter range, the stent preferably expands with the second expansion characteristic.

It is thereby preferably provided that the first expansion characteristic brings about an expansion having a first chronic outward force (COF1) and the second expansion characteristic brings about an expansion having a second chronic outward force (COF2) lower than the first chronic outward force (COF1).

In this manner, a stepped radial force profile can be achieved, enabling expanding of the stent up to the nominal diameter and expanding the corresponding vessel to the nominal diameter, but only allowing adaptive growth of the stent beyond the nominal diameter, said stent applying only a very slight radial force or chronic outward force in said range. The radial force or chronic outward force (COF2) in the second diameter range, that is, in the range beyond the nominal diameter up to the maximum diameter, is preferably selected so that said force is as small as possible and follows the vessel wall during adaptive remodeling. The radial force or second chronic outward force (COF2) in the second diameter range should preferably be selected so that active further expending of the vessel does not occur, but rather the radial force is selected merely such that the stent continues to make close contact with the inner surface of the vessel and follows the same. The stent should thus apply merely such a force in the range greater than the nominal diameter that ensures contact between the stent and the vessel wall. The force should be selected so that the vessel does not need to “pull” on the stent during remodeling, but rather ideally the stent has as little physical effect on the vessel as possible. In other words, instead of or in addition to the first and second expansion characteristic, the stent according to the invention can also be described by means of the first and second radial forces F1, F2 or COF or radial force level to which the radial force drops in the first and second diameter range. Furthermore, the stent according to the invention can be described by means of the radial force curve instead of or in addition to the first and second expansion characteristic, said curve having at least one bend, at least one, preferably two, inflection points, or at least one step. According to the invention, the radial force brought about by the stent drops off severely beyond the nominal diameter and falls to a very low level, preferably such that vessel remodeling does not occur, or occurs only to a very slight degree.

The second diameter range preferably is at least 10% of a maximum diameter Dmax. The second diameter range thus makes up 10% or more of the total expansion. The second diameter range preferably is at least 15%, 20%, 25%, 30%, 40%, 50%, 60%, or 70% of the maximum diameter. It is thereby preferably provided that the second diameter range covers no more than 90% of the maximum diameter Dmax, preferably 80%, further preferably 75%, 70%, 65%, 60%, 55%, or 50%.

The annular segments preferably have a first radial rigidity in a first diameter range less than the nominal diameter, and a second radial rigidity in a second diameter range greater than the nominal diameter. The second radial rigidity is less than the first radial rigidity.

Due to the different first and second radial rigidities, a high radial force is generated at first in the first diameter range and then a lesser radial force in the second diameter range. Rigidity is generally understood to be a ratio of force to deformation. In the first diameter range, the stent has a higher radial rigidity than in the second diameter range, that is, a higher ratio of force to deformation, in this concrete case radial force to radial deformation. When expanding the stent, a higher force per deformation is thereby provided in the first diameter range than in the second diameter range. In the second diameter range, the stent can grow with the vessel during vessel remodeling without applying significant forces to the vessel wall. The radial rigidity is preferably greater than in the second diameter range by at least a factor of 2, further preferably 2.5, further preferably 3, further preferably 4, further preferably 5, further preferably 6, further preferably 7, further preferably 10, further preferably 20.

The stent preferably comprises a first radial force range in the first diameter range up to the nominal diameter and a second radial force range in the second diameter range. The first radial force of the stent is preferably in the first radial force range in the first diameter range, and the second radial force is in the second radial force range in the second diameter range. The stent preferably comprises a first radial force decrement in the first diameter range up to the nominal diameter and a second radial force decrement in the second diameter range. In the first diameter range, the radial force drops from a maximum value from the crimped state to a first plateau to the nominal diameter. When the stent is expanded past the nominal diameter, or when the stent itself expands past the nominal diameter, the radial force drops to a second plateau in a second radial force decrement. When expanding, the radial force asymptotically approaches the first line in the first diameter range and a second line in the second diameter range.

It is further preferable that the radial force drops to a first radial force level in the first diameter range when expanding and drops to a second radial force level, less than the first radial force level, in the second diameter range. The first radial force level is preferably greater than the second radial force level by a factor, wherein the factor is in a range from 2 to 20, preferably in a range from 2 to 10, further preferably from 3 to 10, further preferably from 4 to 10, further preferably from 5 to 9.

In a preferred refinement, a radial force-diameter profile of the stent has a bend or step. A radial force-diameter profile depicts the course of the radial force, starting from a crimped state up to the maximum diameter, graphed against the diameter. For conventional stents, such a radial force-diameter profile does not have a bend or step, but rather the radial force decreases substantially continuously, particularly diminishing, from the crimped state to the maximum diameter and then ends abruptly. The stent according to the invention has a radial force-diameter profile comprising at least one bend, preferably at least two bends. The radial force-diameter profile comprises at least one, preferably two inflection points. The radial force-diameter profile in the first diameter range, when expanding starting from the crimped state, further preferably comprises a first segment having a first slope, then a segment having a second slope, and in the second diameter range, starting from the nominal diameter, comprises a third segment having a third slope and a fourth segment having a fourth slope, wherein the first slope is greater than the second slope and the fourth slope, and the third slope is greater than the second slope and the fourth slope. The third slope can be greater than the first slope. The second slope can be greater than the fourth slope. The first slope is preferably greater than the second slope by a first slope factor, wherein the first slope factor is at least 2.0; 2.5; 3.0; 3.5; 4.0; 4.5; 5.0; 5.5; 6.0; 7.0; 8.0; 9.0; 10.0; 12.0; 15.0. The third slope is preferably greater than the second and/or fourth slope by a second slope factor, wherein the first slope factor is at least 2.0; 2.5; 3.0; 3.5; 4.0; 4.5; 5.0; 5.5; 6.0; 7.0; 8.0; 9.0; 10.0; 12.0; 15.0.

The radial force-diameter profile of the stent, starting from the crimped state to the expanded state, preferably takes the following course: firstly, in a first diameter range, a decrease in slope followed by an increase in slope, then when transitioning into the second diameter range, another decrease in slope and preferably finally another increase in slope. The first decrease in slope in the first diameter range preferably runs up to the nominal diameter. For an expansion beyond the nominal diameter, the first increase in slope then follows, and then a second decrease in slope in the second diameter range. The course can also be described as degressive-progressive-degressive.

The first diameter range further preferably defines a first radial rigidity profile segment and the first radial rigidity profile segment is degressive, regressive, or linear. A degressive or regressive rigidity as the diameter of the stent becomes greater provides a more rapid decrease in force when expanding. The radial force in the first diameter range is preferably substantially constant or only slightly decreasing over at least one segment, and also substantially constant in the second diameter range. This can be achieved in that the radial rigidity is implemented as degressive or regressive.

It is also preferably provided for the second diameter range that the second diameter range defines a second radial rigidity profile segment and the second radial rigidity profile segment is degressive, regressive, or linear.

In a preferred refinement, it is provided that the second radial rigidity profile segment is more degressive or has a greater slope than the first radial rigidity profile segment. In a further preferred embodiment, it is provided that the stent provides a first chronic outward force COF1 in a first radial force range in the first diameter range, and a second chronic outward force COF2 in a second radial force range in the second diameter range. The first chronic outward force is preferably greater than the second chronic outward force by a factor of at least 2, preferably 4, or the second chronic outward force is approximately or nearly 0. The second chronic outward force is preferably 10 N or less. The radial force also depends on the axial length of the stent. The second chronic outward force, expressed as a function of length, is preferably in a range of less than 1 N/mm, preferably less than 0.5 N/mm, further preferably less than 0.25 N/mm. A value of 0.001 N/mm is feasible as a lower limit in each case.

In a preferred refinement, each annular segment comprises a plurality of first circumferential segments and a plurality of second circumferential segments distributed over the annular circumference and structurally differing from each other. In this manner, the different radial forces for the first and second diameter range can be provided by the different first and second circumferential segments. The first and second circumferential segments preferably act in the first diameter range, while the first circumferential segments are fully expanded when the nominal diameter is reached, and only the second circumferential segments continue to act, but only with a lower force, namely the second chronic outward force.

The first circumferential segment are preferably implemented so that said segments have or define the first radial rigidity and the second circumferential segments have or define the second radial rigidity. The second radial rigidity of the second circumferential segments is preferably negligible relative to the first radial rigidity of the first circumferential segments. The first and second circumferential segments preferably joint define the first expansion characteristic in the first diameter range, while solely the second circumferential segments expand in the second diameter range and thus the second expansion characteristic is defined by the second circumferential segments.

It is thereby preferable that the first circumferential segments and second circumferential segments of adjacent annular segments are each axially aligned to each other. That is, first circumferential segments of two adjacent annular segments are axially aligned and thus aligned adjacent to each other. Second circumferential segments of two adjacent annular segments are also axially adjacent and aligned to each other. Uniform opening and expanding of the stent can thereby be achieved without rotary forces acting about a central axis of the stent.

According to a preferred refinement, it is provided that the struts of the first circumferential segments have a greater size than the struts of the second circumferential segments. The first circumferential segments can also be referred to as “hard stent segments” and the second circumferential segments as “soft stent segments.” “Hard stent segments,” “hard circumferential segments,” or “hard annular segments” can be understood to mean such that define the first radial force. “Soft stent segments,” “soft circumferential segments,” or “soft annular segments” can be understood to mean such that define the second radial force. “Hard stent segments” are accordingly such that substantially define the first radial rigidity, and “soft stent segments” are such that substantially define the second radial rigidity.

A stepped radial force profile is particularly achieved by means of a stent of the type described above, wherein the annular segments are formed by elements having different expansion force (hard and soft elements) disposed variously over the annular circumference, of which the hard elements define the nominal diameter for the expansion and the soft elements provide an expansion reserve.

A structure of annular segments of this type made of hard and soft elements is achieved, for example, by means of a stent of the type described above, wherein the annular segments are formed by hard and soft stent segments, of which the hard stent segments define the nominal diameter for the expansion and the soft segments provide an expansion reserve, wherein the struts of the hard stent segments have a greater size than the struts of the soft stent segments.

An annular segment is understood to mean a sequence of struts disposed in a serpentine or zig-zag shape in the expanded state and running transverse to the longitudinal direction of the stent and connected to each other by means of bends or vertices, wherein a plurality of annular segments are preferably disposed adjacent to each other and implement the stent. The annular segments are preferably each connected to adjacent annular segments, preferably by means of connecting struts or by means of common vertices, but also for annular segments on the edge, by direct contact of bends or vertices opposite each other. Connecting struts can be straight or curved in design and can contact the bends of the annular segments at the outside and/or the inside. The terms zig-zag shaped and serpentine are understood to be synonyms.

Stent segments or circumferential segments can be understood in one variant to mean a sequence of struts of equal size running in a serpentine or zig-zag shape when expanded. One annular segment thereby comprises at least one hard stent segment and at least one soft stent segment or one first circumferential segment and one second circumferential segment. Hard stent segments or first circumferential segments are preferably made of a plurality of struts connected to each other by means of bends and spread apart after expansion to the nominal diameter. Soft stent segments or second circumferential segments in one embodiment are preferably made of two struts connected to each other by a bend and running parallel at the nominal diameter and spread apart for a subsequent expansion of the vessel (remodeling) in the diameter range greater than the nominal diameter (expan-sion reserve of the stent). The individual struts of the annular segment preferably run in a straight line and are always connected to adjacent struts by means of bends. In the non-expanded (crimped) state of the stent, all struts run in parallel, as a rule.

The hardness or rigidity of the stent segments can be determined by the size of each of the struts. For example, the strut width defines the rigidity: the wider the struts, the hard or more rigid the stent segment. Soft stent segment can, in the present case, be correspondingly narrower than hard stent segments. Of course, the hardness or rigidity of the stent segments may also be defined by means of the thickness thereof, wherein, however, the strut width is convenient for determining the hardness or rigidity for the typical laser cutting technique, wherein the stent is cut from a tube. In one embodiment example, the difference in strut width gives rise to a stepped radial force profile/expansion profile.

The stents according to the invention preferably comprise two or more second circumferential segments (soft stent segments) per annular segment. The soft stent segments can thereby be disposed linearly successively in the longitudinal direction, or can be offset from each other from annular segment to annular segment in the form of a spiral. The second circumferential segments of two adjacent annular segments are preferably axially adjacent and aligned to each other. The same number of second circumferential segments (soft stent segments) is preferably present in all annular segments. For example, one annular segment comprises two to four second circumferential segments (soft stent segments), for 12 to 18 circumferential segments per annular segment altogether.

In order to establish a further decremented passive expansion behavior, the annular segments may comprise soft stent segments having struts of different sizes. In other words, in one refinement, the second circumferential segments can be designed such that a third expansion characteristic, different from the second expansion characteristic, is provided. Alternatively, third circumferential segments defining such a third expansion characteristic can also be provided. This brings about a subsequent expansion of the stent when the vessel dilates. The different size of struts and general radial rigidity leads to different radial forces and thus to a radial force profile of the stent having a plurality of steps. For example, the soft stent segments have 20% to 60%, preferably 20% to 30% of the width of the hard stent segments. The number of second circumferential segments (soft stent segments) within one annular segment may vary and is based on the particular expansion reserve desired.

The stent according to the invention is preferably made of a shape memory alloy. Said alloy can be a spring steel approved for medical purposes, for example, but is particularly a nickel-titanium alloy such as nitinol. In the present case, the stent is self-expanding.

The stents according to the invention can be produced from a tube in the typical manner by laser cutting. Fundamentally, two or more variants are possible for the soft stent segments (second circumferential segments). For one, the soft stent segments (second circumferential segments) remain fixed and completely closed when shaped. For a vessel dilation beyond the nominal diameter of the stent, said soft segments (second circumferential segments) are opened purely passively (by the draw of the vessel) in the range of the expansion reserve (F2=0). For the other, the soft segments (second circumferential segments) are entirely or partially opened already during shaping. In this case, the soft segments (second circumferential segments) develop a COF (F2>0) in the range of the expansion reserve of the stent enabling the stent to actively grow along beyond the nominal diameter in the range of the expansion reserve thereof. The stent according to the invention is particularly also suitable for vessels undergoing growth in children. It is understood that the hard and soft segments (first and second circumferential segments) may be obtained not only by means of the strut size, but also by means of the strut length and by means of the design and size of the cells of the stents.

It is further understood that the soft segments (second circumferential segments) may be distributed uniformly as well as non-uniformly over the length of the stent. For example, the axial ends of the stent may have a greater or lesser expansion reserve, preferably more or fewer second circumferential segments or differently designed circumferential segments than the center of the stent.

In a further preferred embodiment, the stent comprises large and small cells, wherein the large cells form second circumferential segments (soft stent segments) and the small cells form first circumferential segments (hard stent segments). The terms “small” and “large” relate here to the design of the circumferential segments and are not to be understood as absolute. Large cells differ from small cells particularly in that said large cells have longer sides than the small cells and have a greater cell area in the expanded state than small cells. Large and small cells can also be described by means of the different side lengths, typically formed by struts. Said cells can also be referred to in general as first and second cells. A longer strut generally has a lower rigidity (ratio of force/deformation) than a shorter strut. Said embodiment thus is based on the idea that different chronic outward forces (COF) or radial force levels can be achieved not only by varying the cross section of a strut, but also by varying the free length thereof, whereby a larger cell is achieved. The cells need not all be closed, however, but rather open cells, for example bounded on only two or three sides, are also comprised. Said embodiment is further based on the idea that axially adjacent struts act analogously to springs disposed in parallel or connected in parallel. Large cells, in comparison with small cells, lead to a lesser number of struts disposed adjacent to each other, and thus to fewer spring elements connected in parallel, whereby the radial force in said segments can be reduced.

In a preferred refinement, small cells are disposed in rows adjacent to each other in the axial direction of the stent, are each made of four struts, and are connected to each other by means of the vertices thereof. Large cells are disposed between the rows of small cells, wherein the large cells are each formed of at least six struts. The small cells define the nominal diameter for expansion of the stent and the large cells form an expansion reserve.

The annular segments are preferably disposed so that said segments form a plurality of small and large cells between said segments. The small cells thereby preferably run adjacent to each other and in rows in the axial direction, and are formed by four struts each in one embodiment. Cells adjacent to each other in the axial direction are preferably connected to each other at the vertices thereof, either by means of connecting struts or directly by means of common vertices or nodes.

Large cells are disposed between the annular segments and between the small cells running in the axial direction, and are formed by six or more struts each. Large cells formed by eight or more struts are also preferable.

The small cells implement an expansion matrix defining the nominal diameter for expan-sion. The small cells, due to the small number of struts and area, provide a large radial force essential for vessel expansion. The large cells form an expansion reserve and define the maximum diameter that may follow due to active or passive subsequent expansion in a dilating vessel. Due to the large number of struts/area, said large cells provide a lower radial force, as has been found to be advantageous particularly for passive subsequent expansion, wherein the stent is to follow the dilating vessel.

Of course, small cells may also be disposed between the annular segments and may connect two adjacent annular segments by means of the vertices thereof, for example.

The small cells are thus the hard stent segments (first circumferential segments) having high radial force, and the large cells are the soft stent segments (second circumferential segments) having low radial force.

The large cells are preferably each disposed between the small cells disposed in axial rows, wherein two large cells are preferably disposed between two annular segments. In the axial direction, the large cells may be disposed linearly, preferably axially adjacent to and aligned with each other, or rather offset from each other in a spiral manner.

It is possible by means of forming, particularly by means of partial pre-expansion, to also provide the large cells (soft stent segments) with an active expansion behavior having a lower radial force in comparison with the small cells. This means that, in the case of vessel dilation, the large cells can also actively follow said dilation by means of an intrinsic, low COF.

The invention further relates to the use of stents according to any one of the previously described, preferred embodiment examples for varying the radial force for producing a stepped radial force profile. The invention further relates to the use, that the hard and soft stent segments are formed by struts of different lengths and/or sizes or by cells of different sizes.

In a further aspect, the object indicated above is achieved by a method for producing a stent, preferably a stent according to any one of the previously described, preferred embodiments of a stent according to the first consideration of the invention, having the steps: providing a tube component made of a shape memory material; cutting the stent out of the tube section, wherein the stent comprises first and second circumferential segments; mechanically sealing the second circumferential segments; performing a first forming step by mechanically expanding the first circumferential segments; subsequently releasing the mechanical closure of the second circumferential segments and performing a second forming step by mechanically expanding the second circumferential segments. The method based on the idea that the second circumferential segments develop a lesser radial force than the first circumferential segments. When enlarging the stent in order to bring said stent from the initial cut state into the expanded shape, it is therefore preferable to first form the first circumferential segments by means of a high force, but thereby to close off the second circumferential segments so that the same are not overloaded. In a second step, the closure of the second circumferential segments can then be released and said segment can be elongated by means of a lower force in order to form them. It would also be possible in the alternative to first elongate by means of a low force without closure at first, so that only the second circumferential segments are formed, then to subsequently close the same (either in the expanded state or in a partially crimped state for this purpose) and then to elongate the first circumferential segments by means of a higher force. In both alternatives, however, the second circumferential segments are closed when the first circumferential segments are elongated, and thus are removed from the force flow, so that the force acts exclusively on the first circumferential segments when elongating the same.

The mechanical closing of the second circumferential segments preferably comprises: applying a mechanical retaining means, particularly a clip, for mechanically closing the second circumferential segments. Alternatively preferably, the mechanical closing of the second circumferential segments comprises: introducing material bridges between struts of the second circumferential segments, preferably during the step of cutting. Other closure tech-niques are also conceivable and preferred, such as temporarily applied retaining bands.

Embodiments of the invention are described below using the drawings. Said drawings are not necessarily intended to depict the embodiments to scale, but rather the drawings are schematic and/or slightly distorted as needed for better explanation. With respect to addi-tions to the teachings directly evident from the drawings, reference is made to the relevant prior art. It must thereby be considered that multiple modifications and changes relating to the shape and the detail of an embodiment can be made without deviating from the general idea of the invention. The features of the invention disclosed in the description, in the drawings, and in the claims may be essential to the refinement of the invention both individually and in any arbitrary combination. A combinations of two or more of the features disclosed in the description, the drawings, and/or the claims also fall within the scope of the invention. The general idea of the invention is not limited to the exact shape or the detail of the preferred embodiments shown and described below, nor limited to one subject-matter that would be limited in comparison with the subject-matter claimed in the claims. Where di-mension ranges are given, values within the indicated limits should also be disclosed as limit values and should be able to be applied and claimed arbitrarily. For simplicity, the same reference numerals are used below for identical or similar parts, or for parts having identical or similar functions.

Further advantages, features, and details of the invention arise from the following description of the preferred embodiments and from the drawings; said drawings showing in:

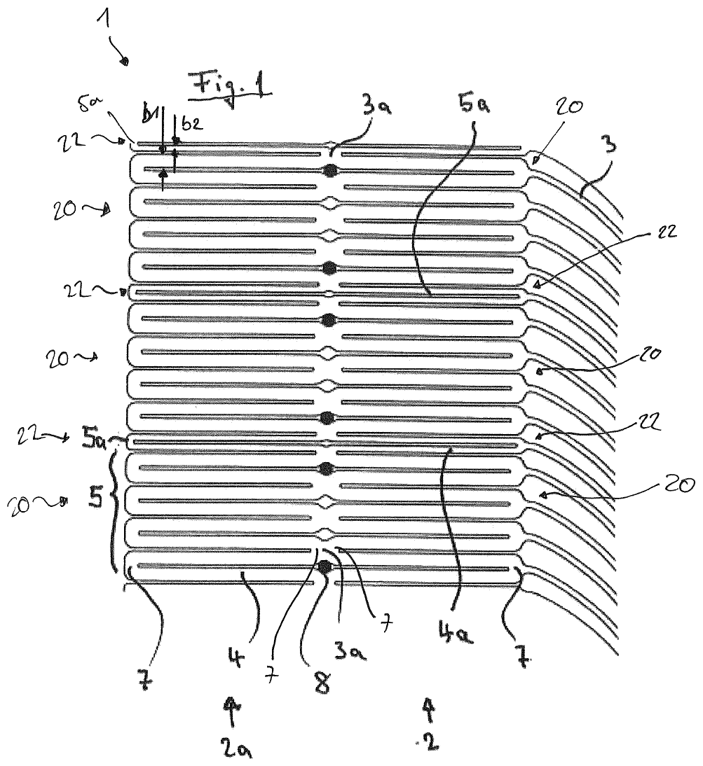

FIG. 1A planar view of the end region of a stent according to the invention,

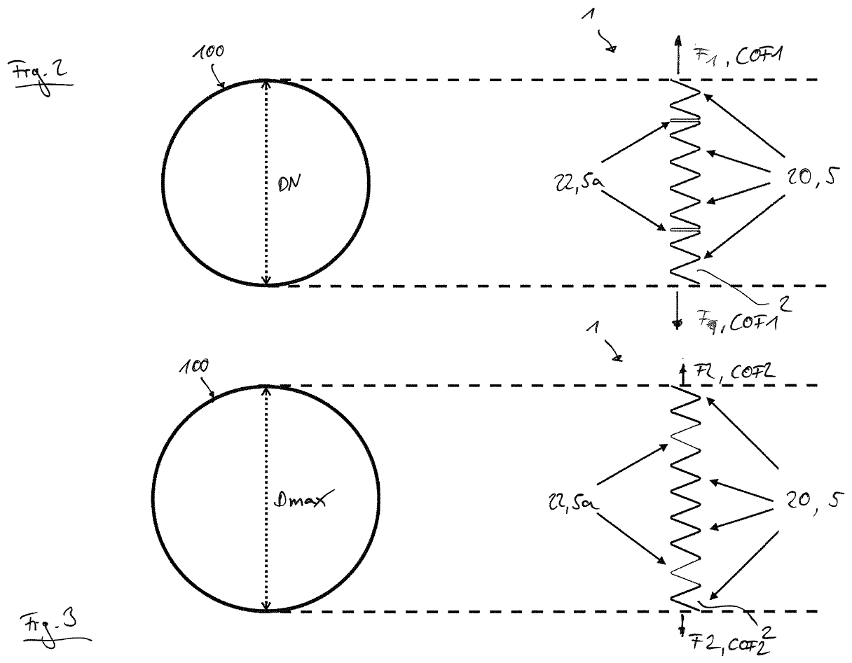

FIG. 2A schematic view of an annular segment of a stent according to the invention in the expanded state,

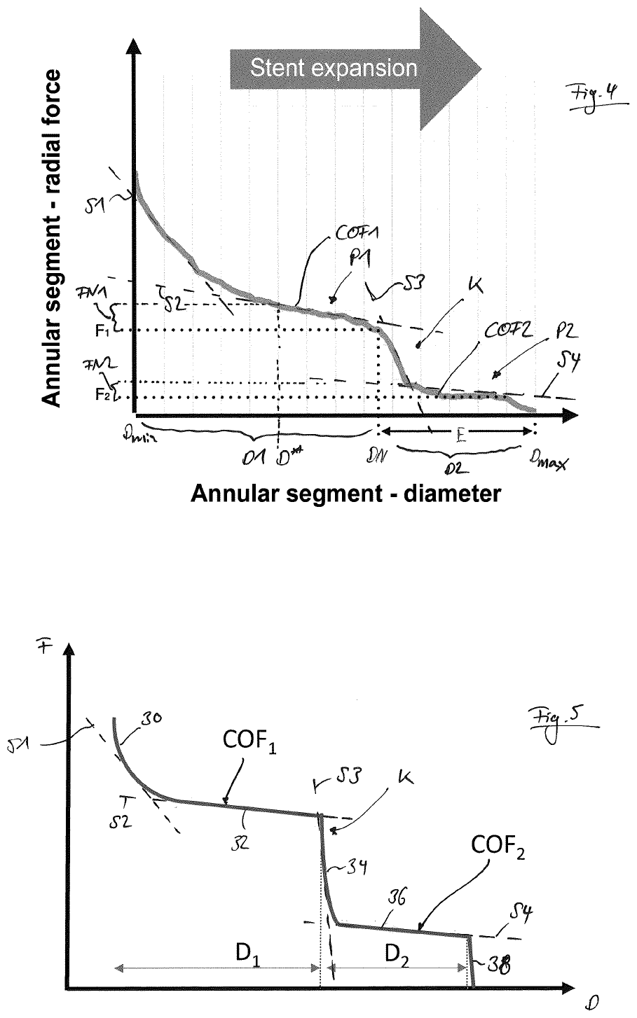

FIG. 3A schematic view of an annular segment of a stent according to the invention in the post-expanded state (expansion reserve),

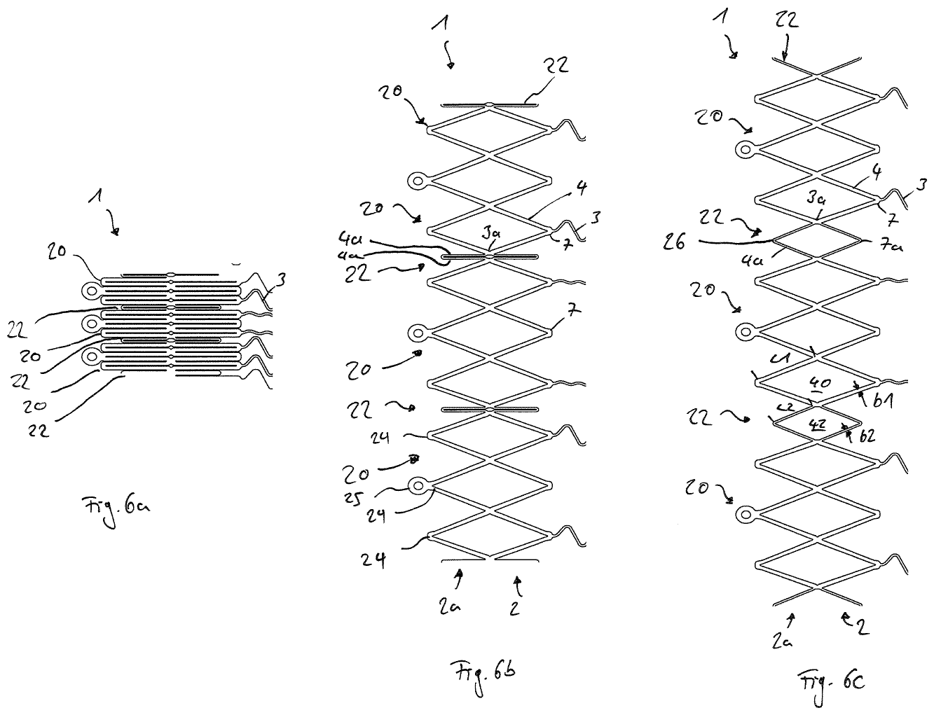

FIG. 4A diagram illustrating the relationship between radial force and expansion/diameter of a stent;

FIG. 5A further diagram illustrating the relationship between radial force and expan-sion/diameter of the stent;

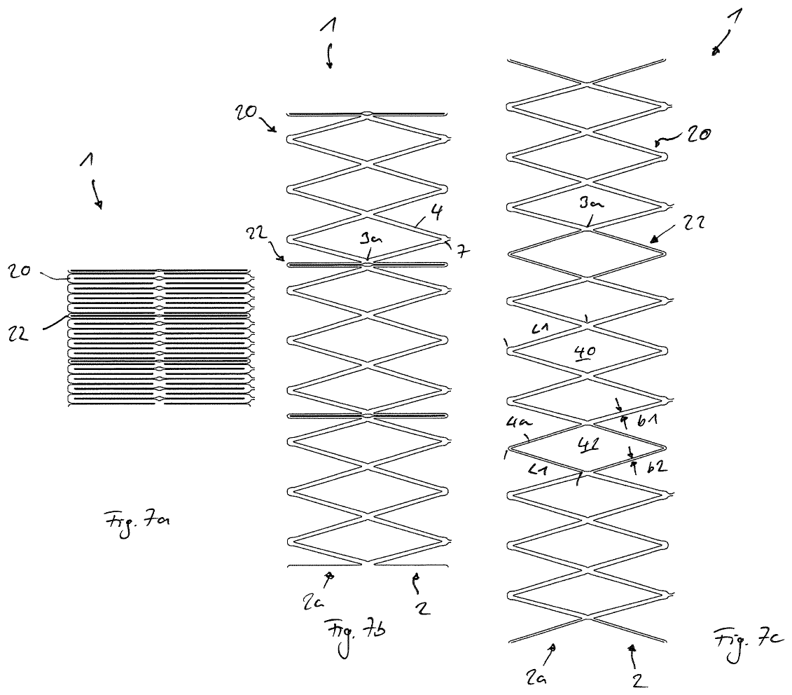

FIGS. 6a to 6c An annular segment of the stent in a crimped state, at nominal diameter, and at maximum diameter;

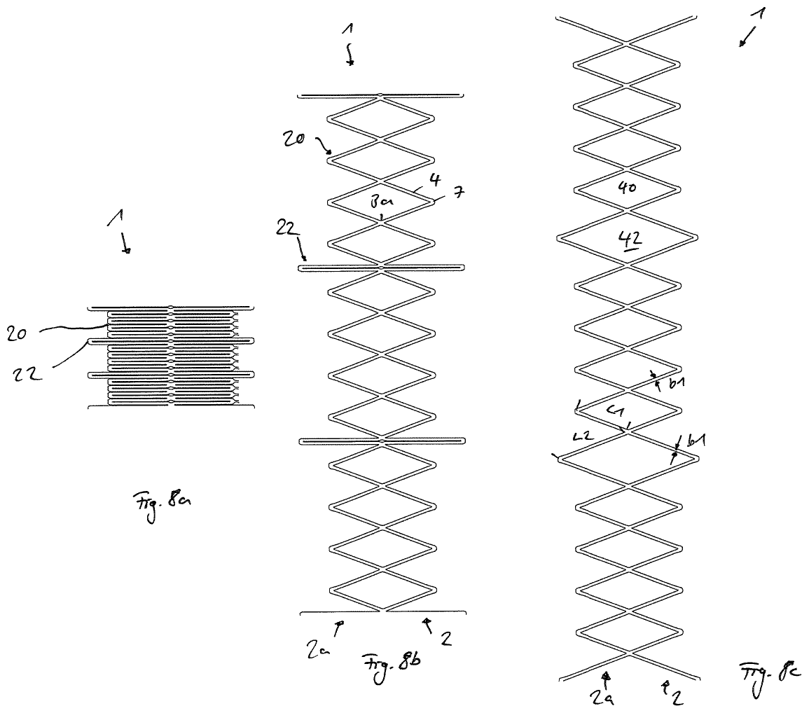

FIGS. 7a to 7c An annular segment of the stent in a further embodiment example in a crimped state, at nominal diameter, and at maximum diameter;

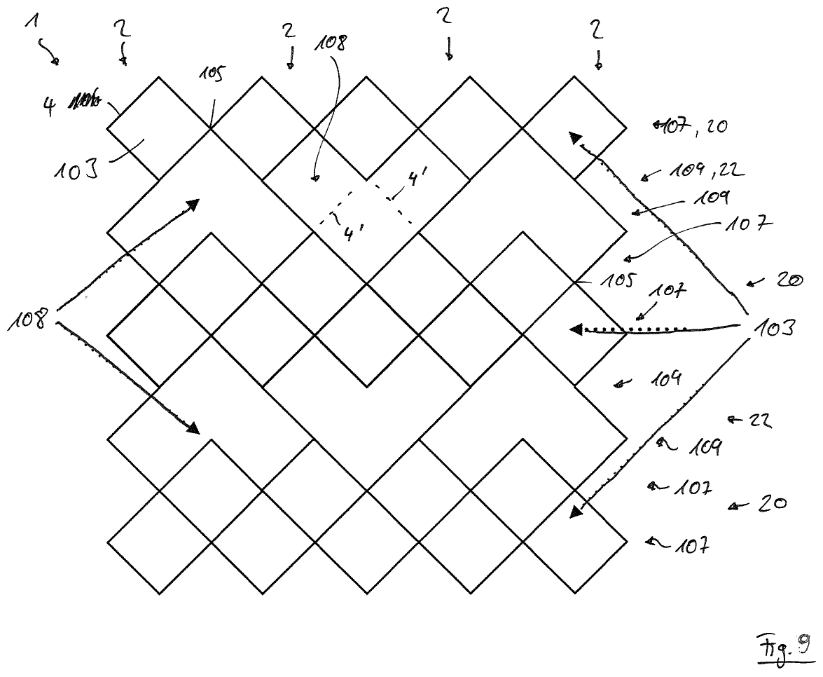

FIGS. 8a to 8c An annular segment of the stent in a further embodiment example in a crimped state, at nominal diameter, and at maximum diameter;

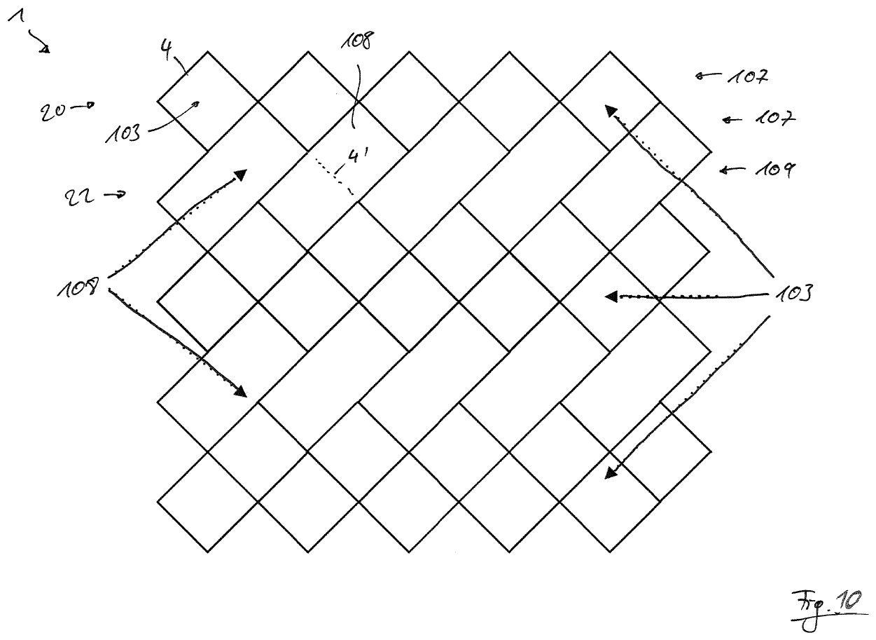

FIG. 9A planar view of a further embodiment example of a stent; and

FIG. 10A planar view of a further embodiment example of a stent.

FIG. 1 shows a planar view of the end region of a non-expanded (self-expanding) stent 1 according to the invention. It is understood that the stent 1 has a generally tubular structure, but is shown here for better clarity in a planar view, that is, only the surface of the otherwise tubular stent is shown.

In the embodiment shown in FIG. 1, the stent 1 has two annular segments 2, 2a disposed axially (horizontally with respect to FIG. 1) adjacent to each other and next to each other. The individual annular segments 2 and 2a are connected to the adjacent annular segments 2 and 2a, here by means of connecting struts 3. That is, with respect to FIG. 1, one or more annular segments (not shown) may be present to the right of the annular segment 2 and connected to the annular segment 2 by means of the connecting struts 3. The connecting struts 3 primarily ensure the axial alignment of the individual annular segments 2, 2a, but do not themselves bring about any or any substantial radial force. In the stent segment shown, the annular segment 2a at the edge is further connected to the adjacent annular segment 2 directly at contact points 3a of the bends 7 axially adjacent and opposite each other. The individual struts 4, 4a of the annular segments 2 and 2a have a serpentine shape, so that said segments spread apart in a zig-zag shape during expansion. All struts 4 and 4a are connected to the adjacent struts 4 and 4a thereof by bends 7.

The stent 1 is implemented so that the annular segments 2, 2a comprise a first expansion characteristic in a first diameter range D1 (cf. FIG. 4, 5) less than the nominal diameter DN and comprise a second expansion characteristic in a second diameter range D2 greater than the nominal diameter DN, wherein the first and the second expansion characteristics are different from each other. In this manner, the stent 1 is preferably able to provide a first chronic outward force COF1, preferably a first radial force in a first range, in the first diameter range D1, and a second chronic outward force COF2, preferably a second radial force in a second range, in the second diameter range D2. This is described below in more detail and with reference to the FIGS. 2-5.

In the first embodiment example shown in FIG. 1, each of the annular segments 2, 2a has a plurality of circumferential segments, here a total of three first circumferential segments 20 and three second circumferential segments 22, each disposed alternately about the circumference of the corresponding annular segment 2, 2a. In this sense, the second circumferential segments 22 are distributed uniformly about the circumference, in the present case offset from each other by 120°. A uniform distribution is advantageous in order to achieve uniform expanding of the stent 1. However, more or fewer than the three first and three second circumferential segments 20, 22 each may be provided. In particular, a stent 1 having only one second circumferential segment 22 can also be according to the invention. In further embodiments, one or more third circumferential segments (not shown) may also be provided in order to achieve further stepping of the radial force.

In the stent 1 shown in FIG. 1, six struts 4a of a lesser width b2 are disposed in each annular segment 2 and 2a. Said struts 4a of lesser width b2 implement the second circumferential segments 22, also referred to as “soft stent segments 5a,” in pairs. The first circumferential segments 20, also referred to as “hard stent segments 5,” comprise eight struts 4 or four pairs of struts in the embodiment example shown here. The struts 4 of the first circumferential segments 20 have a first width b2. For the normal expansion from a crimped state to the nominal diameter, only the first circumferential segments 20 (hard stent segments 5) are spread apart, while the second circumferential segments 22 (soft stent segments 5a) remain in the closed state thereof.

In the case shown, the struts 4a of the second circumferential segments 22 (soft stent segments 5a) are about half the width of the struts 4 of the first circumferential segments (hard stent segments 5). The force required for spreading open the second circumferential segments 22 (soft stent segments 5a) is correspondingly less and can by implication be applied by a dilating vessel (passive expansion). Alternatively, the second circumferential segments 22 (soft stent segments) can have an expansion reserve due to the shape thereof for enabling active expansion as the vessel dilates. The thickness in the radial direction and the length of the individual struts 4, 4a in the present embodiment example are equal for all struts 4, 4a. The stent 1 is preferably cut from a uniform raw material. The first width b1 of the first struts 4 of the first circumferential segment 20 preferably defines a first radial rigidity, and the second width b2 of the second struts 4a of the second circumferential segments 22 preferably defines a second radial rigidity. In the embodiment example of FIG. 1, the radial rigidity, as well as a radial force-diameter profile, is therefore determined by the shape and design of the struts 4, 4a and can be adjusted by means of the same. The first width b1 of the first struts 4 of the first circumferential segment 20 is preferably greater by a factor than the second width b2 of the second struts 4a of the second circumferential segments 22. A ratio b1/b2 is preferably in a range from 1.5 to 5, preferably 1.5 to 4, further preferably 2 to 3.5.

The bends 7 are the attachment points of the connecting struts 3 and the rotation or bend-ing points for spreading or crimping a self-expanding stent 1.

The points 8 shown are the mounting points at which the stent 1 is mounted to a carrier during forming so that the soft stent segments 5a remain closed at the nominal diameter.

A central region, not shown, of the stent 1 may connect to the right end region shown by means of the connecting struts 3 and may have a conventional design except for the soft stent segments 5a. The stent 1 may also be made of only the two annular segments 2, 2a and thus not comprise any connecting struts 3. It is also conceivable that one or more further annular segments are connected directly by means of contact points 3a to the right axial end, with respect to FIG. 1, instead of the connecting struts 3, and may be identical or similar in design to the annular segments 2, 2a.

FIG. 2 shows a schematic view of a stent 1 at the nominal diameter DN thereof after implantation in a vessel 100. The depicted annular segment 2 shows the first circumferential segments 20 (hard stent segments 5) spread apart in the open state, while the second circumferential segments 22 (soft stent segments 5a) are still closed. F1 indicates the high radial forces of the first circumferential segments 20 (hard stent segments 5).

In comparison with the embodiment example of FIG. 1, only one annular segment 2 is shown in each of the FIGS. 2 and 3. It should be understood, however, that the embodiment example of FIGS. 2 and 3 may also comprise at least two annular segments 2, 2a. In this respect, the depiction having only one annular segment 2 in FIGS. 2, 3 particularly serves for illustrative purposes. FIG. 1, however, shows the crimped state of the stent 1, in which said stent is compressed to a minimum diameter Dmin (cf. FIG. 4-6), in order to be implanted at said minimum diameter Dmin in the crimped state. In the crimped state (cf. FIG. 1), the individual struts 4, 4a are substantially parallel to each other. In the expanded state, the struts 4, 4a are at an angle to each other and encompass a space or cells between said struts.

For the expansion to the nominal diameter DN (FIG. 2), only the first circumferential segments 20 are fully expanded, while the second circumferential segments 22 are still closed or in a partially crimped state. In the fully expanded state of the first circumferential segments 20, the first circumferential segments 20 do not exert and further radial force. A radial rigidity of the second circumferential segments is preferably selected so that a second radial force F2 able to be exerted by the second circumferential segments 22 is substantially less than a first radial force F1 exerted by the first circumferential segments 20. In addition, the second radial rigidity of the second circumferential segments 22 is selected so that the second radial force F2 exerted by the same is so low that no expanding of the vessel 100 occurs. In this respect, the second circumferential segments 22 remain closed up to the nominal diameter DN of the first circumferential segments 20; the second circumferential segments 22 cannot expand against the first radial force F1 developed by the first circumferential segments 20 because the second radial force F2 developed by the same is too low.

FIG. 3 shows a schematic view of the stent 1 at the maximum diameter Dmax thereof after dilation of the vessel 100 and full expansion of the first circumferential segments 20. The depicted annular segment 2 shows the first circumferential segments 20 (hard stent segments 5) spread apart in the open state, as shown in FIG. 2, and the second circumferential segments 22 (soft stent segments 5a) also spread apart, having followed the vessel dilation. F2 indicates the low radial forces responsible for the subsequent expansion of the second circumferential segments 22 (soft stent segments).

After the stent 1 has been expanded to the nominal diameter DN by the radial force developed by the first circumferential segments 20 and second circumferential segments 22, and thereby the vessel 100 has optionally dilated somewhat in diameter under application of a first chronic outward force COF1, a further deflection of the vessel wall and ultimately further dilation of the vessel 100 may still occur (known as remodeling). The stent 1 makes it possible to still apply a force in a diameter range D2 greater than the nominal diameter DN, namely a second chronic outward force (radial force) COF2. In the embodiment example shown in FIGS. 1 through 3, said effect is implemented by means of the two circumferential segments 22 comprising only such a rigidity (second radial rigidity) as to develop the second radial force F2. Said force is selected to be so low that further dilating or remodeling of the vessel 100 is prevented. The second radial force F2 is preferably selected in a range enabling the stent 1 to only follow the vessel wall.

At the maximum diameter Dmax, the second circumferential segments 22 are also fully expanded. For the case that the second circumferential segments 22 are designed such that said segments develop a radial force F2=0, the maximum diameter is particularly defined by the diameter at which a radial force acts inwardly when exceeded, that is, that would have to draw the vessel 100 toward the stent 1 in case of further remodeling. For the case that the second circumferential segments 22 are designed such that said segments develop a radial force F2>0, the maximum diameter is particularly defined by the diameter at which the stent 1 is in a relaxed position. When exceeded here, in turn, a radial force would need to act inwardly, that is, the vessel 100 would have to draw on the stent 1 in case of further remodeling. However, the second radial rigidity of the second circumferential segments 22 is preferably selected such that the vessel wall is not influenced, neither by a substantial outward radial force nor by a substantial inward radial force. Enlarging of the vessel 100 beyond the nominal diameter DN is thus possible, even without loosening the stent 1, and contraction of the vessel 100 to less than the nominal diameter DN is effectively prevented.

FIG. 4 shows the radial force curve of a stent 1 according to FIG. 1 as a function of the degree of expansion or diameter. After implantation, the stent 1, having a self-expanding design, expands to the nominal diameter DN as the radial force decreases and exerts a first radial force F1 in a first range. The nominal diameter is reached at DN. The maximum diameter Dmax is achieved subsequently when remodeling the vessel 100. For said subsequent expansion, the reduced second radial force F2 acts in a second radial force range. The range E indicates the expansion reserve.

The first chronic outward force COF1 is defined here by the range in which the stent 1 will or may make contact with the vessel 100 when expanding. In FIG. 4, a force F1 is exerted precisely at the nominal diameter DN. But if the vessel 100 is somewhat smaller than the nominal diameter DN, then a slightly greater radial force is exerted. The stent 1 is to be designed so that said stent makes contact with the vessel in a region in which the first chronic outward force COF1 is at a first plateau P1, that is, between the diameter D* and the nominal diameter DN. In said region, the first chronic outward force COF1 is in a first radial force level FN1.

From the minimum diameter range Dmin in the crimped state of the stent 1, the radial force exerted drops quickly until said force reaches a level in the range of a first radial force level FN1 able to be treated as approximately constant, about halfway through the first diameter range D1 (at D*) between the minimum diameter Dmin and the nominal diameter DN. Up to this point, the radial force drops degressively. The stent 1 has only one first expansion characteristic up to this point, namely a degressive one. The vessel 100 is radially expanded by the force of the stent 1, up to the nominal diameter DN. It is also evident from FIG. 4 that the radial force drops in a stepwise manner after the nominal diameter DN, into the range of a second radial force level FN2, and particularly to the second radial force F2 also able to be treated as substantially constant in turn in the range of the second radial force level FN2. The stent 1 can thus exert a second chronic outward force COF2. In the second diameter range D2, the radial force thus again drops off degressively and the stent 1 accordingly has a second expansion characteristic. Said characteristic is different from the first, as the degree of drop and the force level achieved is different. The second chronic outward force COF2 provided is so low that the vessel 100 is no longer actively expanded by the stent 1, but rather the stent 1 simply gently follows any remodeling of the vessel 100 with no substantial force. The second diameter range D2, greater than the nominal diameter DN, is thus an expansion reserve E. As can also be seen in FIG. 4, the radial force-diameter curve has a bend K1 or step in the range of the nominal diameter DN. In said range, the curve no longer runs continuously and asymptotically, as is known from conventional stents, but rather drops off suddenly to the second radial force level FN2. In the embodiment example shown in FIG. 4, the first radial force F1 is about 5 to 6 times greater than the second radial force F2.

Furthermore, slopes S1 through S4 are drawn as straight lines in FIG. 4. In the first diameter range D1, the radial force asymptotically approaches the line S2, indicating a second slope S2. In the second diameter range D2, the radial force asymptotically approaches the line S4, indicating a fourth slope S4. Both the second and the fourth slope S2, S4 are small and can be substantially treated as linear. Starting from the crimped state at Dmin, the radial force drops severely at first with a slope S1, drawn tangentially here, and then drops with the second slope S2 from the diameter D* to the nominal diameter DN. The first slope S1 is significantly greater than the second slope S2. For an expansion beyond the nominal diameter DN, the radial force then drops again at first with a greater slope S3, and then drops again with a lesser slope S4.

FIG. 5 shows a further radial force-diameter curve of the stent 1 according to the invention. Again from the minimum diameter Dmin in the crimped state of the stent 1, the radial force, drawn on the ordinate, drops degressively at first in a first segment 30, until the radial force reaches a first plateau 32 in the first diameter range D1. At said first plateau 32, corresponding in the embodiment example of FIG. 4 to the first radial force level FN1, it is not absolutely necessary that a constant force be exerted across the first diameter range D1, even if such is desirable. The first chronic outward force COF1 generated in said range remains approximately constant when expanding up to the nominal diameter DN, dropping slightly with the slope S2. When expanding the stent 1 beyond the nominal diameter DN, the radial force then abruptly drops in a step 34 with the third slope S3, which need not necessarily be linear, but can also be degressive. It is preferable that the force decrement is as steep as possible and the radial force drops from the level of the first chronic outward force COF1 to a substantially low level as immediately as possible when exceeding the nominal diameter DN. In the embodiment example shown in FIG. 5, the radial force drops to a second plateau 36, representing here again a substantially constant radial force analogous to the first plateau 32, referred to here as a second chronic outward force COF2. The level of the second chronic outward force COF2 remains substantially constant across the second diameter range D2, and drops slightly with the fourth slope S4. When the maximum diameter Dmax is reached, the radial force then drops to 0. This occurs in the embodiment example shown here in a step 38, in turn, but could also run out in a more flat, linear, or degressive manner. The first chronic outward force COF1 in the example shown in FIG. 5 is 3 to 3.5 times the second chronic outward force COF2.

FIGS. 6a through 6c illustrate a further embodiment example of a stent 1. All three figures show the circumference of the stent 1 in an unrolled depiction. It should be understood, however, that the stent 1 is actually annular in shape and so the end shown at the top in each of the FIGS. 6a through 6c is connected to the end shown at the bottom in each case. The depictions can also be understood as cutting patterns for a laser cutting method. In FIG. 6A, the stent 1 is shown in the crimped state at the minimum diameter Dmin. FIG. 6a shows the stent 1 at the nominal diameter DN and FIG. 6c at the maximum diameter Dmax, that is, in the fully expanded state.

Just as in the first embodiment example of FIG. 1, the stent 1 according to the FIGS. 6a through 6c has three first circumferential segments 20 and three second circumferential segments 22. The second circumferential segments 22 each in turn have two second struts 4a per annular segment 2, 2a connected to each other by means of a second bend 7a. The first circumferential segments 20 each have six first struts 4 per annular segment 2, 2a. The six first struts 4 form three first prongs 24 in each first circumferential segment 20 (see FIG. 6b), of which the center prong is optionally provided with an anchor 25, implemented here as a ring. The second struts 4a together form a second prong 26. The first width b1 of the first struts 4 is, in turn, significantly greater than the second width b2 of the second struts 4a, by approximately a factor of 3 here.

In contrast to the first embodiment example of FIG. 1, the second struts 4a of the second circumferential segments 22 have a different length than the first struts 4 of the first circumferential segments 20. Concretely, the second struts 4a have a second length L2, while the first struts 4 have a first length L1. In the present embodiment example (FIG. 6a through 6c), the second length L2 is shorter than the first length L1. This can slightly increase the rigidity, but limits the maximum diameter Dmax, which may also be advantageous in order to prevent excessive remodeling.

The first struts 4 of the adjacent annular segments 2, 2a are each disposed in opposite orientations, so that said struts form first cells 40. In an analogous manner, the second struts 4a of the adjacent annular segments 2, 2a implement second cells 42. Because the second struts 4a are shorter here than the first struts 4, the second cells 42 are smaller than the first cells 40, here by approximately 1/3.

FIGS. 7a through 7c illustrate the stent 1 in a further embodiment example in the same manner as FIGS. 6a through 6c. Identical and similar elements are labeled with the same reference numerals as in FIGS. 6a through 6c, and the differences from the embodiment example of FIGS. 6a through 6c are described substantially below.

In the embodiment example according to FIG. 7a through 7c, all struts, the first struts 4 and the second struts 4a, have the same length, here L1. The different rigidities and thus also radial forces are implemented in the present embodiment example exclusively by means of the width of the corresponding struts. The first struts 4 have a first width b1 and the second struts 4a have a second width b2. Because all struts have the same length, the first and second cells 40, 42 formed by the struts are also substantially identical.

FIGS. 8a through 8c illustrate the stent 1 in a further embodiment example in the same manner as FIGS. 6a through 6c. Identical and similar elements are labeled with the same reference numerals as in FIGS. 6a through 6c, and the differences from the embodiment example of FIGS. 6a through 6c are described substantially below.

In contrast to the embodiment example of FIGS. 6a through 6c, all struts in the embodiment example of FIGS. 8a through 8c, the first struts 4 and the second struts 4a, have the same strut width, here b1. The different rigidity and resulting radial force is implemented in the present embodiment example solely by means of the length of the struts. While the first struts 4 have a length L1, the second struts 4a have a relatively increased length L2. Due to the thus shortened lever acting on the bends 3, 3a, the rigidity of the second circumferential segments 22 is reduced. A ratio L2/L1 is approximately 3/2 here, but may also be selected as greater or lesser, depending on the application. Different cell sizes of the first and second cells 40, 42 also arise thereby. In this respect, the present embodiment example can also be described in that the second circumferential segments 22 form larger cells than the first circumferential segments 20.

A further difference from the embodiment examples of FIGS. 6a through 6c and 7a through 7c is that the first circumferential segments 20 each comprise four pairs of first struts 4 and thus also four first cells 40, while in the embodiment examples of FIGS. 6a through 6c and 7a through 7c, only three are provided in each case.

FIGS. 9 and 10 show alternative embodiment examples of the stent 1 having a stepped radial force profile. In contrast to the preceding embodiment examples, however, all struts are implemented having the same width and same cross section.

FIG. 9 shows a stent 1 according to the invention as a principle sketch in the expanded state, that is, again only the circumferential surface. The stent 1 is annular and the central axis runs horizontally in FIG. 9. The circumference is formed by serpentine annular segments 2 implementing a sequence of small cells 103 in the axial direction by means of struts 4 connected to vertices 105. In the axial direction, the stent 1 is defined by small cells 103 of four struts 4 each disposed in three rows 107 in the depicted case.

In contrast to the preceding embodiment examples, therefore, annular segments 2 are not formed by a continuous zig-zag shape of struts 4, but rather struts 4 are selectively removed from the complete network of struts 4 having prongs 24, 26. For a regular zig-zag shape, cells are formed of four struts 4 each, as is the case for the cell 103 at the top left corner of FIG. 9. Such cells are also provided in the preceding embodiment examples.

Large cells 108 are disposed between each of the annular segments 2 and the rows 107, and in the depicted case are formed by eight struts 4 each. The large cells 108 are implemented at an angle or L-shaped and the size thereof corresponds to three small cells 103 disposed at an angle. In another embodiment variant, it is entirely possible to implement the large cells 108 as rectangles, hexagons, or squares and thereby to vary the radial force. In any case, further small cells 103 may be disposed between the rows 107 of small cells 103 and have influence on the radial force of the stent 1.

The large cells 108 are formed in that individual struts 4 are left out of the regular, generally rhombic, basic pattern. Two eliminated struts 4′ are indicated by dashed lines for a large cell 108 in the middle.

The stepped radial force profile comes about in the embodiment example shown here as follows: the individual struts 4 adjacent to each other in the axial direction act as spring elements connected in parallel in the circumferential direction and thus in the direction of the radial force. Ten axially adjacent struts 4, that is, connected in parallel, are provided in each of the complete rows 107 in the embodiment example shown here. In incomplete rows 109, only four struts 4 adjacent in the axial direction are provided. The spring force provided by the struts 4 is thereby reduced by 60%, so that different chronic outward forces COF can be generated in this manner.

In the embodiment example of FIG. 9, two adjacent, complete rows 107 form a first circumferential segment 20, and two incomplete rows 109 form a second circumferential segment 22. FIG. 10 shows a variant of the stent 1 from FIG. 9 having smaller large cells 108 relative to FIG. 9. The large cells 108 are each bounded by four struts 4 of equal length. Each annular segment 2 comprises the same number of large cells 108, two in the depicted case.

In a complete row 107, ten struts 4 are provided here in turn, while a total of six struts 4 are provided in incomplete rows 109. This means that the spring force of the struts 4 connected in parallel is reduced by only 40% here, wherein a higher second chronic outward force COF2 is achieved in comparison with FIG. 9.

A stent 1 according to the invention is preferably cut out of a tube section. The tube section is preferably made of a nitinol material and has shape memory properties. FIGS. 1, 6a, 7a, 8a show not only the crimped state, but also the initial state after cutting, preferably laser cutting. This means that when producing the stent 1, said stent is cut out of the tube section in the initial state shown in FIGS. 1, 6a, 7a, and 8a. A plurality of forming steps are then performed. To this end, the second circumferential segments are mechanically closed at first, and the first circumferential segments are mechanically released. The stent 1 is then expanded by means of a tool. Because the second circumferential segments are mechanically closed, only the first circumferential segments are expanded and formed. After said first forming step, the stent 1 has the configuration shown in FIGS. 6b, 7b, and 8b. The mechanical closing of the second circumferential segments is then removed. The stent 1 is then further expanded by means of the same tool or a further tool. The second circumferential segments are then thereby substantially formed. The force required to this end is significantly less than the force required for expanding the first circumferential segments.

The mechanical closure can act in a clamping, that is, frictional, positive, and/or adhesive manner. Clips labeled as 8 in FIG. 1 form a mechanical closure for the second circumferential segments. Alternatively or in addition, material bridges can be provided between the struts of the second circumferential segments 22 for opposing expansion of the second circumferential segments in the first forming step. Said material bridges must then be removed prior to performing the second forming step.

1. A self-expanding, flexible intravascular stent for use in a vessel, the stent comprising:
at least two annular segments connected axially to each other, each of the at least two annular segments formed of a plurality of struts connected to each other in a meandering manner,
wherein the stent is configured to transition from a crimped state in which the stent has a reduced diameter to an expanded state in which the stent has a nominal diameter, and
wherein the at least two annular segments have a first expansion characteristic in a first diameter range that is smaller than the nominal diameter, and a second expansion characteristic, different from the first expansion characteristic, in a second diameter range that is greater than the nominal diameter.

2. The stent of claim 1,
wherein the second diameter range is at least 10% of a maximum diameter.

3. The stent of claim 1, wherein:
the at least two annular segments have a first radial rigidity in the first diameter range, and a second radial rigidity in the second diameter range, and
the second radial rigidity is less than the first radial rigidity.

4. The stent of claim 1, wherein the at least two annular segments have a first radial force decrement in the first diameter range up to the nominal diameter and a second radial force decrement in the second diameter range.

5. The stent of claim 4, wherein the at least two annular segments have a first radial force level in the first diameter range a second radial force level that is less than the first radial force level, in the second diameter range.

6. The stent of claim 1, wherein a radial force-diameter profile of the stent comprises a bend or a step.

7. The stent of claim 1, wherein a radial force-diameter profile of the stent comprises:
a first segment having a first slope in the first diameter range, when the stent is expanding starting from the crimped state,
a second segment having a second slope in the second diameter range, starting from the nominal diameter,
a third segment having a third slope, and
a fourth segment having a fourth slope,
wherein the first slope is greater than the second slope and the fourth slope, and
wherein the third slope is greater than the second slope and the fourth slope.

8. The stent of claim 1, wherein a radial force-diameter profile of the stent comprises:
a decrease in slope followed by an increase in slope in the first diameter range, and another decrease in slope and another increase in slope in the second diameter range.

9. The stent of claim 1, wherein a radial force-diameter profile of the stent has at least one, inflection point.

10. The stent of claim 1, wherein the first diameter range defines a first radial rigidity profile segment that is degressive or linear, and the second diameter range defines a second radial rigidity profile segment that is degressive or linear.

11. (canceled)

12. The stent of claim 10, wherein the second radial rigidity profile segment is more degressive or has a greater slope than the first radial rigidity profile segment.

13. The stent of claim 1, wherein the stent provides a first chronic outward force in a first radial force range in the first diameter range, and a second chronic outward force in a second radial force range in the second diameter range, and wherein the first chronic outward force is at least twice as great as the second chronic outward force or the second chronic outward force is about 0.

14. The stent of claim 1, wherein each of the at least two annular segments comprises a plurality of first circumferential segments and a plurality of second circumferential segments that are different from the plurality of first circumferential segments.

15. The stent of claim 14, wherein the plurality of first circumferential segments and the plurality of second circumferential segments together define a first chronic outward force in the first diameter range, and the plurality of second circumferential segments define a second chronic outward force in the second diameter range.

16. The stent of claim 14, wherein the plurality of first circumferential segments and the plurality of second circumferential segments are each axially aligned to each other.

17. The stent of claim 14, wherein first struts of the plurality of first circumferential segments are larger than second struts of the plurality of second circumferential segments.

18. The stent of claim 17, wherein rigidity of the first struts and the second struts is based on strut width, such that the first struts have a first strut width and the second struts have a second strut width that is less than the first strut width.

19. The stent of claim 14, wherein the stent further comprises two or more of the second circumferential segments in each of the at least two annular segments.

20. The stent of claim 14, wherein the plurality of second circumferential segments are disposed spirally offset over the length of the stent.

21. The stent of claim 14, wherein the plurality of second circumferential segments are disposed linearly in the axial direction of the stent.

22. The stent of claim 1, wherein said stent is made of a shape-memory alloy.

23. The stent of claim 22, wherein said stent is made of a nickel-titanium alloy.

24. The stent of claim 1, wherein the at least two annular segments are connected to adjacent annular segments via connecting struts.

25. (canceled)

26. The stent of claim 1, wherein at least one outer annular segments is directly connected to an adjacent annular segment.

27. (canceled)

28. The stent of claim 9, wherein;
the at least two annular segments form a plurality of cells,
the plurality of cells comprise large cells and small cells,
the small cells form first circumferential segments, and
the large cells form second circumferential segments.

29. The stent of claim 28, wherein;
the small cells are disposed in rows adjacent to each other in the axial direction of the stent, are each formed from four struts, and are connected to each other via vertices,
the large cells are disposed between the rows of the small cells, are each formed from at least six struts,
the small cells define the nominal diameter when the stent is the expanded state, and
the large cells form an expansion reserve.

30. (canceled)

31. The stent of claim 28, wherein two or more large cells are disposed between the at least two annular segments.

32. The stent of claim 28, wherein the large cells are disposed spirally offset over the length of the stent.

33. The stent of claim 28, wherein the large cells are disposed linearly in the axial direction of the stent.

34. The stent of claim 28, wherein the plurality of struts have a uniform length.

35. (canceled)

36. A method for producing a stent, the method comprising:
providing a tube section made of a shape memory material;
cutting the stent out of the tube section, wherein the stent comprises first circumferential segments and second circumferential segments;
mechanically closing the second circumferential segments;
mechanically expanding the first circumferential segments; and
releasing the mechanical closure of the second circumferential segments and mechanically expanding the second circumferential segments.

37. The method of claim 36, wherein the mechanical closing of the second circumferential segments comprises:
applying a retainer to the second circumferential segments.

38. The method of claim 36, wherein the mechanical closing of the second circumferential segments comprises:
introducing material bridges between struts of the second circumferential segments.
